(19) United States
(12) Patent Application Publication
(10) Pub. No.: US 2004/0024238 A1
(43) Pub. Date: Feb. 5, 2004

(54) Use of nitriles as polar aprotic solvents

(76) Inventors: Nathalie Laurain, Irigny (FR); Samir Zard, Gif-sur-Yvette (FR)

Correspondence Address: Norman H. Stepno, BURNS, DOANE, SWECKER & MATHIS, L.L.P., P.O. Box 1404, Alexandria, VA, 22313-1404, US

(21) Appl. No.: 10/342,327

(22) Filed: Aug. 20, 2003

Related U.S. Application Data
(63) Continuation of application No. 10/019,186, filed on Mar. 12, 2002, now abandoned.
Application No. 10/019,186 is a 371 of international application No. PCT/FR00/01864, filed on Jun. 30, 2000.

(30) Foreign Application Priority Data
Jul. 2, 1999 (FR) 99/08558

(51) Int. Cl. C07C 253/00

(52) U.S. Cl. 558/342

(57) ABSTRACT

The invention concerns the use of aliphatic nitriles with relatively high molecular weight, as polar aprotic solvents, in particular in nucleophilic substitution reactions of the aromatic type. Said nitriles have a molecular mass more 79, preferably more than 90. The invention is useful for synthesis of fluorinated aromatic compounds.

A subject matter of the present invention is the use, as polar aprotic solvents, of aliphatic nitrites with relatively high molecular weights.

A more particular subject matter of the invention is the use of these nitrites as solvents in nucleophilic substitution reactions, in particular aromatic nucleophilic substitution reactions.

The present invention also relates to novel reactants of use for exchange between fluorine and halogens or halogens resulting in the fluorination of a molecule.

Most polar aprotic solvents are expensive solvents which are difficult to access and which require storage conditions during use which are relatively restrictive.

Thus, in the specific case of chlorine/fluorine exchange, one of the most efficient solvents is sulfolane, which is expensive and which requires purification measures to be taken before use, in particular to prevent it from incorporating too much water.

In addition, sulfolane is not supposed to be particularly healthy.

The problem is particularly acute in the case of the use of these solvents in the synthesis of fluorinated derivatives. These derivatives are increasingly used in the pharmaceutical or agrochemical industry because of their properties, which differ significantly from the properties of conventional halogenated derivatives.

Fluorinated derivatives are difficult to access, in particular because of the reactivity of fluorine, which is such that it is impossible to easily obtain fluorinated derivatives in a direct manner.

Consequently, one of the most widely employed techniques for manufacturing fluorinated derivatives consists in reacting a halogenated or pseudohalogenated derivative, generally a chlorinated derivative, in order to exchange the halogen with an inorganic fluorine, generally an alkali metal fluoride. The alkali metal advantageously having a high atomic weight (generally at least equal to that of potassium).

On the industrial scale, the most widely used fluoride is potassium fluoride, which constitutes a satisfactory economic compromise between cost and effectiveness.

This technique is used both for exchange on aliphatic carbons (that is to say, carbons with sp³ hybridization) and on aromatic or vinyl carbons (carbons with sp² hybridization).

This halogen exchange technique is used in particular for exchanges on aromatic rings, whether homocyclic or heterocyclic.

Exchange in the case of chlorine and fluorine, as is more generally the case with Aromatic Nucleophilic Substitutions (SN_{Ar}), is usually extremely slow and difficult, except in exceptional cases where the aromatic ring is highly depleted in electrons. Furthermore, the reaction is extremely sensitive to impurities and in particular to water, which results in the formation of an aromatic ether in place of the expected fluorinated derivatives.

This etherification reaction is not the only side reaction, as the products obtained are often unstable products. This instability extends to the “Meisenheimer” complex, which is specific to “aromatic” nucleophilic substitution reactions.

In addition, to cap it all, the reaction has to be carried out at high temperatures (that is to say, markedly greater than 100° C.), often in the vicinity of 200° C., which implies the use of solvents and catalysts which are highly resistant to these temperatures.

This is why one of the aims of the present invention is to provide a technique which results in the use of reactants and solvents which are simpler than those used previously.

Another aim of the present invention is to provide a technique which, applied to alkali metal fluorides, indeed even to alkaline earth metal fluorides, makes it possible to significantly increase their reaction kinetics.

Another aim of the present invention is to provide a technique which makes it possible to increase the yields of the reaction. These yields are essentially the reaction yield, or RY, and the conversion yield, or CY, which is the image of the selectivity of the reaction.

Another aim of the present invention is to provide solvents which are easy to access, which are not very complex and which have good chemical and thermal resistance.

Another aim of the present invention is to provide solvents which facilitate exchange reactions between halogens and/or pseudohalogens and in particular SN₁, SN₂ and in particular SN_{Ar} nucleophilic substitution reactions.

Another aim of the present invention is to provide reactants capable of promoting or producing exchanges by nucleophilic substitution, in particular bimolecular nucleophilic substitutions and aromatic nucleophilic substitutions.

These aims and others which will become apparent subsequently are achieved by means of the use, as polar aprotic solvent, of a nitrile with a molecular mass of greater than 79, advantageously of greater than 90.

This use as polar aprotic solvent is particularly advantageous in the case of nucleophilic substitution reactions, in particular aromatic nucleophilic substitution reactions.

The nitrile is advantageously a compound comprising at least one nitrile functional group of aliphatic nature, that is to say that the carbon carrying the or a nitrile functional group has sp³ hybridization.

It is desirable, when the carbon carrying the nitrile functional group carries another electron-withdrawing functional group, that is to say a functional group with a Hammett constant σₚ greater than 0.2, for the carbon not to carry hydrogen. It is preferable for this restriction to be applied as soon as the Hammett constant σₚ is greater than 0.

For further details on Hammett constants, reference may be made to the third edition of the handbook “Advanced Organic Chemistry” (pages 242 to 250) written by Professor Jerry March and published by John Wiley and Sons.

It is desirable for the nitrile constituting the solvent, or a portion of the solvent, to be such that, by the most direct route, two electron-withdrawing functional groups (generally nitrile functional groups) are separated by at least two carbons, advantageously three carbons, preferably four carbons. This is particularly the case when the nitrile is a dinitrile. In the case in particular where the electron-withdrawing functional groups are separated by only two, indeed even three, carbons, it is preferable for the carbon or carbons carrying the electron-withdrawing functional groups and in particular the nitrile functional group, or nitrile functional groups, to carry an electron-donating group, for example of the alkyl type (taken in its etymological meaning of an alkyl alcohol from which the OH functional group has been removed), including the aralkyl type.

When the nitrile is an aliphatic polynitrile, a significant effect is already obtained when at least one of the carbons carrying the nitrile functional group carries an electron-donating group or functional group. This can be placed on a scale by the ratio of the alpha hydrogens to the total hydrogens.

Of course, among the population of the carbons carrying a nitrile functional group, the greater the proportion of carbon also carrying an electron-donating functional group, the better the selectivity. However, the kinetics then become less favorable.

It is preferable for the electron-donating groups to be chosen from those which possess little steric hindrance; when they are alkyls, methyl is preferred to primary alkyls and the latter to secondary alkyls.

For the notion of electron-withdrawing and electron-donating groups, reference will be made to the work of general chemistry, Advanced Organic Chemistry, Reactions, Mechanisms, and Structure, 3rd edition; by Jerry March, edited by John Wiley & Sons, in particular to chapter 9 and more particularly to pages 242 to 248. The sign of the Hammett constant, as it appears in particular in table 4 on page 244, will be consulted in assessing whether a group is electron-withdrawing or electron-donating.

According to the present invention, it is preferable for the optional electron-withdrawing groups present on the solvent molecule to be less electron-withdrawing than the nitrile group, preferably at most equal to the trifluoromethyl groups, more preferably to the value 0.40.

The electron-donating groups are advantageously groups which are electron-donating via an inductive effect.

It is desirable for the number of hydrogens in the alpha position with respect to an electron-withdrawing group with a σₚ in the region of that of the nitrile, that is to say with a Hammett constant σₚ at least equal to 0.4, to be such that the ratio of the number of hydrogens in the alpha position to the total number of hydrogens (H_{α}/H_{T}) is less than or equal to ⅔, advantageously less than or equal to ½, preferably less than or equal to 40%, more preferably less than or equal to 30%.

The molecular mass related to the aliphatic nitrile functional group is at least equal to 40, advantageously to 47, preferably to 54. This value is obtained by the total molecular mass divided by the number of aliphatic nitrile functional groups, that is to say the number of nitrile functional groups carried by a carbon atom possessing sp³ hybridization.

It is preferable for the melting point of said nitriles to be less than 100° C., preferably less than 50° C., more preferably less than 30° C.

It is preferable for the boiling point of said nitrile to be at least equal to 150° C., preferably at least equal to 200° C. The nitrites can be used alone or as a mixture with other solvents. However, in the latter case, it is desirable either for them to be mixed with other nitriles-according to the present invention or for them to be greatly predominant by mass with respect to the other solvents. Thus, it is preferable, when a nitrile according to the invention is mixed with a solvent which is not another nitrile according to the present invention, for the percentage of nitrile by mass with respect to the combined solvents to be at least equal to 50%, preferably to ⅔, more preferably to ¾.

It is advisable, when solvents other than the nitrites according to the present invention are used, to use solvents which are relatively low in polarity, that is to say solvents with a relative dielectric constant ε at most equal to 20, preferably at most equal to approximately 10 (the 0 symbols are not regarded as significant figures). It is also preferable for the donor number, defined as the ΔH (variation in enthalpy), expressed in kilocalories, of the combination of the dipolar aprotic solvent with antimony pentachloride, to be at most equal to approximately 10.

Mention may be made, as solvent capable of being mixed with the nitriles according to the invention, of those exhibiting a relatively high boiling point, that is to say a boiling point at least equal to 130° C., preferably to 150° C.-200° C., and derivatives of the chloroaromatic type, indeed even anisole.

It is also preferable for the solubility of water in these solvents capable of being mixed with the nitrites according to the invention to be as low as possible, preferably less than 15%, preferably less than 10%, preferably less than 5%. It should be noted that. some of the nitriles according to the invention are capable of dissolving significant amounts of water (up to the order of 10%, indeed even 20%, by mass) but they still exhibit a very low hygroscopicity. This makes it possible to easily remove water possibly present as impurity.

For economic reasons, it is preferable to use, as nitrile, those which comprise, as functional group, only nitrile functional groups.

The preferred formula of the nitrites according to the present invention is of the following type:

A-L-C(R₁) (R₂)—C≡N

in which R₁ and R₂, which are alike or different, are groups, advantageously hydrocarbonaceous groups, which are donors or which are weakly electron-withdrawing, that is to say with a σₚ of less than 0.10, or else a hydrogen (it should be pointed out that, as hydrogen is the reference group, it is neither donating nor withdrawing and its σₚ is by definition equal to 0).

Advantageously, R₁ and/or R₂ are alkyl or hydrogen. When they are alkyl, it is preferable for their number of carbons to be at most equal to 6, advantageously to 4.

L is an arm chosen from single bonds or an alkylene group, optionally interrupted by ether functional groups resulting from epoxides, as is specified later, and in particular polymethylene. It advantageously comprises at most 10 carbon atoms, preferably at most 8. L advantageously does not comprise any functional groups.

A can be hydrogen, aryl or alkyl, advantageously with a number of carbons at most equal to 6. A can also be a group:

—C(R₃) (R₄)—CN

where R₃ and R₄, which are alike or different, have the same values as R₁ and R₂, with the same preferences.

Without this being preferred, the arm L can comprise other groups of the A type but the number of groups of A type is limited by the above restrictions and those relating to the melting points of said nitrile. This is because, to act as solvent, the nitrile has to be molten and relatively low in viscosity at the temperature of use. It is preferable for there to be at most 3, advantageously at most 2, preferably at most only 1, group A.

The viscosity, expressed in millipascal.second, at the temperature of use should be at most equal to 500 mPa.s, advantageously at most equal to 100 mPa.s, more preferably to 50 mPa.s.

It is preferable for these values to be achieved at temperatures at most equal to 150° C., advantageously at most equal to 100° C.

Although this is not preferred, the group L can comprise several links formed from an ethylene oxide or propylene oxide group; more specifically, these are groups resulting from the polycondensation, more precisely from the oligocondensation, of ethylene oxide and/or propylene oxide.

The total number of carbons in the nitrites according to the invention is advantageously at most equal to 20, preferably at most equal to 10.

The present invention is also targeted at compositions of use as reactants or as media of use for SNA reactions and in particular for exchanges between halogen and in particular between fluorine and halogen with a higher atomic rank than fluorine.

These compositions comprise, as solvent, a nitrile according to the present invention and additionally comprise a phase transfer agent, either of the cryptand type, such as crown ethers, or of the onium or inium type. Oniums are compounds with a name in the nomenclature comprising as, generally as suffix, the sequence of letters “onium”. They are compounds of a semimetal, in particular from the nitrogen column or from the sulfur column, which are sufficiently substituted to carry a positive charge. Thus, the atoms of the nitrogen column, when they are substituted four times by a hydrocarbonaceous radical, constitute oniums. Thus, quaternary ammoniums or quaternary phosphoniums can be used as phase transfer agent. Likewise, sulfoniums (tertiary in their case) also constitute phase transfer agents but the latter are less advantageous as they are relatively more unstable than the others. “Iniums”, the affix of which is “inium”, result from oniums by replacing two hydrocarbonaceous groups by a single hydrocarbonaceous group, the latter then being connected to the semimetal via a double bond (for example pyridinium). “Oniums” are preferred to “iniums”.

The oniums used as phase transfer agent are known to a person skilled in the art.

Tetraalkylammoniums and tetraalkyl- or tetraphenylphosphoniums are the most commonly used. However, the latter exhibit the disadvantage of being relatively expensive.

To avoid beta-elimination, tetramethylammonium is the most commonly used, although it is relatively unstable from approximately 150° C.; mention may also be made of benzyltrimethylammonium. Finally, use may be made, as phase transfer agent, of salts of alkali metals, particularly heavy alkali metals, such as cesium and rubidium. However, these compounds are relatively toxic and in particular very expensive, which limits their use.

The amount of phase transfer agent is generally between 0.1% and 20%, preferably between 1 and 5%, of the mass of solvent used. However, the upper limit can easily be exceeded and only represents a problem of cost. If it is expressed with respect to the substrate, a value of between 0.1 and 10 molar % is the most common.

Thus, the oniums are chosen from the group of the cations formed by the elements from groups VB and VIB (as defined in the Periodic Table of the Elements published in the supplement to the Bulletin de la Société chimique de France in January 1966, with respectively four or three hydrocarbonaceous chains).

To carry out the exchange between fluorine and halogens with a higher atomic rank, it is appropriate to have a fluoride source. This fluoride source can be alkali metal fluorides or alkaline earth metal fluorides but much more preferably alkali metal fluorides. Generally, a potassium fluoride is used.

Thus, it is desirable for the solid in suspension to exhibit a particle size such that its d₉₀, defined as being the mesh size which allows 90% (by mass of the solid) to pass, is at most equal to 100 μm, advantageously at most equal to 100 μm, preferably at most equal to 20 μm. The low limit is advantageously such that the d₉₀ of said solid in suspension is [lacuna] least equal to 0.1 μm, preferably at least equal to 1 μm. It [lacuna] sometimes simpler to measure only the d₅₀. In this case, it may be indicated that the d₅₀ is advantageously at most equal to 20 micrometers.

Generally, the ratio of said alkali metal fluoride to said substrate is between 1 and 1.5, preferably in the vicinity of 1.25, of stoichiometry (with respect to the halogens to be exchanged). However, this ratio is generally not critical provided that it makes possible suitable agitation of the reactant. Except for some aliphatic exchange reaction, it is recommended for [lacuna] water content of the reactant to be at most equal to approximately 2%, preferably 1%, by mass with respect to the mass of the reactant. As is known to a person skilled in the art for other solvents, it is preferable, for SN_{Ar} substitutions, to remove as completely as possible water possibly present in the reactant and to drop to values of 1⁰/₀₀ and even of 0.1⁰/₀₀.

The temperature of the exchange is advantageously between 100 and 250° C., preferably between 120 and 200° C.

When the substrates are volatile, the desired product can be distilled as it is formed by using the property of fluorinated derivatives, which are in the great majority of cases more volatile than their chlorinated, brominated or iodinated homologues.

The substrates are advantageously aryl heavy halides (that is to say, halides with an atomic weight greater than that of fluorine). To obtain good yields, it is preferable to use aryls with an electron richness which has been significantly depleted. This depletion can be due either to the presence of a heteroatom in the (six-membered) aromatic ring, such as, for example, in pyridine or quinoline.

Very clearly, the electron depletion can also be brought about by the presence of electron-withdrawing groups. Electron impoverishment can be due to both these causes. To obtain good exchange, it is preferable for the sum of the σₚ values on a six-membered aromatic homocyclic ring to be at least equal to 0.4.

Mention may be made, among the electron-withdrawing groups which, by themselves alone, create conditions for good exchange, of the nitro, perfluoroalkyl or nitrile groups and derivatives of acid functional groups. The most advantageous substrates are substrates with a pyridine ring and substrates with a phenyl ring. In the examples of the invention, dichloronitrobenzene is used as paradigm, teaching, for example, the exchange techniques according to the present invention, which can be transposed to all the aromatic substrates capable of being subjected to exchange with fluorine.

According to one of the preferred forms of the present invention, the exchange, advantageously the SN_{Ar} exchange, is carried out under the action of microwaves and in the presence of cesium. The combination of the microwaves and of the nitrile solvents according to the present invention, in particular in the presence of cesium, gives very good results. The best results are those obtained when at least most, advantageously ⅔, preferably ⅘ and even at least 90% of the fluoride is introduced in the form of cesium fluoride. To obtain a competitive process, it is then advisable to provide for the reprocessing of the cesium salts.

The reaction is generally carried out at a temperature below that used for a conventional reaction, that is to say without the actinic activation according to the present invention.

It is preferable to carry out the reaction under actinic activation at a temperature of at least 10° C., advantageously 20° C., preferably 40° C., below that of the temperature limit conventionally accepted for said solvent used.

According to one of the possible, indeed even preferred, forms of the present invention, the microwaves are emitted over short periods (from 10 seconds to 15 min), alternating with cooling phases. The respective durations of the microwave emission periods and of the cooling periods are chosen so that the temperature at the end of each microwave emission period remains below a set initial temperature which is generally below that of the resistance of the ingredients of the reaction mixture.

It is also possible to carry out the invention according to a procedure in which the reaction mixture is subjected simultaneously to microwaves and to cooling. According to this alternative form, the power given off by the microwaves is then chosen so that, for a set initial temperature, generally the operating temperature, it is equivalent to the energy removed by the cooling system, apart from the heat given off or absorbed by the reaction.

The claimed activation process furthermore has the advantage of being compatible with a continuous operating method. This method of use advantageously makes it possible no longer to have problems of heat exchanges capable of being generated during operations in which the reactor, in which the microwaves are emitted, is opened and closed. According to this operating method, the materials to be activated are introduced continuously via an inlet orifice into the reactor, where they are subjected to activation by microwaves, and the activated products are continuously discharged from said reactor via an outlet orifice.

The most volatile compounds can also be recovered continuously as they are formed. This recovery can be carried out, for example, by distillation.

According to a favored form of the invention, the use is recommended of a power given off by the microwaves of between 1 and 50 watts per milli-equivalent of fluoride ions. Likewise, the microwaves are preferably used at a frequency of 300 MHz to 3 GHz. The frequency used is generally 2.45 GHz and the associated wavelength is in the region of 12 cm in air; the penetration of the electromagnetic field can vary between 2 and 10 cm according to the size of the losses.

It is also desirable to submit to the constraint according to which the power given off by the microwaves is between 2 and 100 watts per gram of reaction mixture.

As was mentioned previously, the presence of a phase transfer catalyst is useful, indeed even necessary, for the satisfactory progression of the reaction. The best phase transfer catalysts which can be used are generally oniums, that is to say these are organic cations in which the charge is carried by a semimetal. However, when cesium fluoride is used as main fluorine source, it is pointless to provide phase transfer catalysts other than cesium.

During the study which led to the present invention, it was shown that the action of the microwaves on the oniums and the iniums in the presence of a large amount of fluorides was extremely harmful to the survival of this phase transfer catalyst.

According to the present invention, it has been shown that the presence of anions which are less aggressive with respect to the oniums, such as, for example, chloride, makes possible the stabilization of said onium.

Thus, it is noticed, for example, that, during a chlorine/fluorine exchange reaction, the stability of the onium increases as the reaction progresses since this reaction gives off chloride anions.

More generally, it is preferable to make sure that, during the reaction, the presence of an anion distinct from the fluorides is ensured in amounts greater than one times, advantageously than two times, preferably than three times, the amount in equivalents of said unstable onium.

As was mentioned previously, the chloride ion is a good candidate for reducing the decomposition of the oniums during the reaction.

According to a favored form of the invention, when the phase transfer catalyst is an onium which is unstable in the presence of fluorides, the reaction is carried out in the presence of chlorides in an amount greater than one times the amount in equivalents of said unstable onium.

This instability of the oniums makes it even more advantageous to operate without onium but using cesium as sole phase transfer agent.

As was mentioned previously, the alkali metal fluoride or alkaline earth metal fluoride is at least partially present in the form of a solid phase.

The fluoride is advantageously a fluoride of an alkali metal with an atomic number at least equal to that of sodium and is preferably a potassium fluoride or a cesium fluoride.

It should be pointed out that complex fluorides of KHF₂ type also appear among the fluorides which can be used. However, the use of fluorides not carrying a hydrogen atom will be favored as they are only rarely suitable for aromatic substitutions.

In the present description, the chlorine/fluorine exchange reaction, sometimes known in the field under the acronym “halex” (halogen exchange), is used as paradigmatic example of the reactions employing alkali metal fluorides.

According to the present invention, it is also possible, when the incoming product is more volatile than the substrate and more volatile than the solvent, to remove the target derivatives as they are formed.

This makes it possible to avoid excessively long residence times under particularly severe conditions.

In addition, this makes it possible to exploit the law of mass action.

A person skilled in the art can infer, from the examples which follow, that, in terms of activity, the branched dinitriles give better results than the mononitrile. With regard to selectivity, the mononitrile is better than the branched dinitrile, itself better than the [lacuna] dinitrile.

The examples which follow illustrate the invention.

### EXAMPLES

### General Procedure for the Examples

The potassium fluoride, the 2,4-dichloronitrobenzene and the solvent are charged to a Schott tube or reactor placed under a nitrogen atmosphere and then heating is carried out at the temperature indicated for 4 h. After 4 h, the reaction mixture is subsequently filtered and analyzed by gas chromatography.

### Example 1

In this example, the general procedure was used under the following conditions: dichloronitrobenzene (DCNB) with the amount of potassium fluoride stoichiometrically necessary for a double exchange (in fact, a slight excess equal to 1.05 SA) and an amount of tetramethylammonium chloride (TMAC) equal to 0.04 times the amount of dichloronitrobenzene, expressed in moles. The figures between brackets located before the name of the chemical product express its molar ratio, the substrate being taken as reference.

(1) DCNB+(2) KF+(0.04) TMAC+(3) Solvent/170° C., 4 h, Schott tubes

| | | DC_{DCNB} | RY_{4C2FNB} | RY_{DFNB} | Σ CY |
|---|---|---|---|---|---|
| Solvent | B. p. (%) | (%) | % | % | % |
| Sulfolane* | | 99 | 13 | 57 | 71 |
| 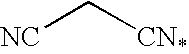 | 220 | 46 | 1 | 0 | 2 |
| 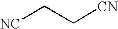 | 265-267 | 66 | 7 | 1 | 12 |
| 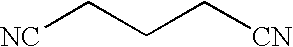 | 285-257 | 98 | 17 | 43 | 62 |
| 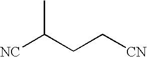 | 269-271 | 98 | 17 | 58 | 76 |
| 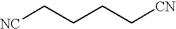 | 295 | 99 | 13 | 52 | 70 |
| 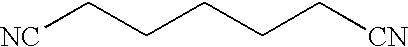 | 175-176/14 mm | 97 | 20 | 48 | 70 |
| 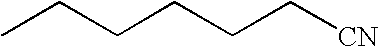 | 186-187° C. | 38 | 30 | 5 | 91 |
| 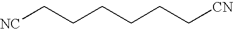 | 185/15 mm | 98 | 15 | 46 | 63 |
| 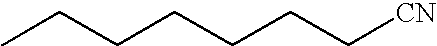 | 198-200 | 82 | 44 | 26 | 87 |
| 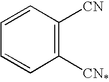 | M. p. 139-141° C. | 58 | 32 | 8 | 70 |
| 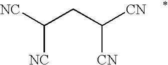 | | 5 | 0 | 0 | 0 |
| 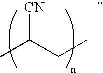 | | 6 | 0 | 0 | 0 |
| 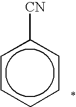 | 188 | 71 | 46 | 23 | 97 |

*comparative

### Example 2

Test Under Microwaves in Adiponitrile

The test molecule is in this instance DCNB with solely cesium fluoride.

Characteristics of the equipment used for the text:

the reactor is made of quartz and has a volume of 40 ml

the stirrer is made of glass

the maximum power of the generator is 300 W.

Temperature regulation is manual and not automatic; the power of the microwave radiation is adjusted manually as a function of time to keep the temperature constant. All the compounds are introduced at ambient temperature; the microwave radiation is switched on and operates at full power (P=300 W) until the desired temperature is achieved. The power of the generator is then adjusted in order to keep the medium at the desired temperature.

The molar ratios with respect to DCNB are as follows:

| | | | | RY | RY | |
|---|---|---|---|---|---|---|
| | Temp. | Time | DC | CFNB | DFNB | □ CY |
| Solvent | (° C.) | (min) | (%) | (%) | (%) | (%) |
| ADN | 180° C. | 10 min | 51 | 43 | 8 | 100 |
| ADN | 200° C. | 10 min | 72 | 50 | 22 | 100 |
| ADN | 220° C. | 10 min | 96 | 32 | 54 | 90 |

DCNB = 1
Solvent adiponitrile (ADN) = 3
CsF = 2.1

1. The use as polar aprotic solvent in nucleophilic substitution reactions of a nitrile with a molecular mass of greater than 79, preferably of greater than 90, and in that said nitrile comprises at least one nitrile functional group carried by an aliphatic carbon (that is to say, with sp³ hybridization), with the condition that, when the aliphatic carbons carry, in addition to a nitrile functional group, an electron-withdrawing functional group with a Hammett constant σₚ of greater than or equal to 0.2, it does not carry hydrogen.

2. The use as claimed in claim 1, characterized in that, when the nitrile exhibits 2 electron-withdrawing functional groups (including the nitrile), these 2 electron-withdrawing functional groups will be separated by at least 2 carbon atoms, advantageously 3, preferably 4.

3. The use as claimed in claim 2, characterized in that the two electron-withdrawing functional groups are nitrites.

4. The use as claimed in claims 1 to 3, characterized in that, when the nitrile exhibits 2 electron-withdrawing functional groups (including the nitrile), these 2 electron-withdrawing functional groups will be separated by at least 2 carbon atoms, advantageously 3, preferably 4.

5. The use as claimed in claims 1 to 4, characterized in that, when the aliphatic carbons carry, in addition to a nitrile functional group, an electron-withdrawing functional group with a Hammett constant σₚ of greater than or equal to 0.05, it does not carry hydrogen.

6. The use as claimed in claims 1 to 5, characterized in that the number of hydrogen in the alpha position with respect to an electron-withdrawing group with σₚ is greater than or equal to 0.4 is such that the H_{α}/H_{T} ratio is at most equal to ⅔, advantageously to ½, preferably to 40% and more preferably to 30%.

7. The use as claimed in claims 1 to 6, characterized in that the molecular mass, with respect to the number of aliphatic nitrile functional groups, is at least equal to 40, advantageously to 47, preferably to 54.

8. The use as claimed in claims 1 to 7, characterized in that the nucleophilic substitution reaction is carried out under activation by microwaves.

9. The use as claimed in claims 1 to 7, characterized in that the nucleophilic substitution reaction is an aromatic substitution reaction.

10. The use as claimed in claims 1 to 8, characterized in that the nucleophilic substitution reaction is a chlorine fluorine exchange aromatic substitution reaction.

11. The use as claimed in claims 1 to 8, characterized in that the nucleophilic substitution reaction is a chlorine fluorine exchange aromatic substitution reaction in the presence of cesium fluoride.

12. A composition of use as chlorine/fluorine exchange reactant, characterized in that it comprises, for successive or simultaneous addition, a nitrile as defined in claims 1 to 5, an agent which is a source of fluoride ions chosen from alkali metal fluorides or alkaline earth metal fluorides, and optionally a phase transfer agent, with the condition that, when the concentration of phase transfer agent is less than 0.5% by mass of the nitrile, the latter represents at least 50%, preferably ⅔, by mass of the mass of the solvents used.

13. The composition as claimed in claim 12, characterized in that it comprises cesium as phase transfer agent.

14. The composition as claimed in claims 12 and 13, characterized in that it predominantly comprises (>50% as fluorine equivalent) cesium fluoride as alkali metal fluoride.

15. The composition as claimed in claims 12 to 14, characterized in that said nitrile is chosen from aliphatic dinitriles.

16. The composition as claimed in claims 12 to 14, characterized in that the nitrites are chosen from pentanenitrile, hexanenitrile, octanenitrile, glutaronitrile, methylglutaronitrile, adiponitrile, pimelonitrile or suberonitrile.
